(19) United States
(12) Patent Application Publication
(10) Pub. No.: US 2025/0306194 A1
(43) Pub. Date: Oct. 2, 2025

(54) WIRELESS-ENABLED MICROMOTION DETECTION

(71) Applicant: Intel Corporation, Santa Clara, CA (US)

(72) Inventors: Maximilian PINAROC, San Francisco, CA (US); Cagri TANRIOVER, Portland, OR (US); Hong LU, San ]ose, CA (US); Rahul SHAH, San Francisco, CA (US); Tal ILANI, Eshchar (IL); Assaf Gurevitz, Ramat Hasharo (IL); Udi APSEL, Sitria (IL); Uri PERLMUTTER, Holon (IL)

(21) Appl. No.: 18/617,676

(22) Filed: Mar. 27, 2024

(51) Int. Cl. G01S 13/56; G01S 7/00; G01S 7/41

(52) U.S. Cl. CPC G01S 13/56; G01S 7/006; G01S 7/415

(57) ABSTRACT

Disclosed herein are systems, devices, and apparatuses for wireless-enabled micromotion sensing. The wireless-enabled micromotion sensing system causes a transmit antenna to wirelessly transmit a series of probe transmissions and causes a receive antenna to wirelessly receive a series of reflected signals from the probe transmissions, where each reflected signal of the series of reflected signals corresponds to a corresponding transmission of the series of probe transmissions. The wireless-enabled micromotion sensing system determines a characteristic dataset comprising a change in a channel characteristic over time as between the series of reflected signals and the series of probe transmissions. The wireless-enabled micromotion sensing system determines based on the characteristic dataset whether the series of reflected signals is indicative of a periodic micromotion of an object.

### TECHNICAL FIELD

The disclosure relates generally to wireless sensing systems, and in particular, to wireless systems that may detect periodic micromotions of nearby objects, such as a person's breathing, based on wirelessly transmitted/received signals.

### BACKGROUND

In general, wireless transmissions from a transmitter to a receiver may be impacted by objects in the environment. As wireless signals propagate from the transmitter to the receiver, they may bounce off of objects, causing constructive and/or destructive interference of the signal, especially where the transmitter and/or receiver are moving. More specifically, wireless sensing systems may assess the impact objects may have on wireless transmissions to determine whether an object is within the signal path between transmitter and/or receiver. This is the concept behind classic radar systems, where a radio signal is transmitted and its back reflections may be observed by a receiver to determine whether an object exists, its size, its shape, its composition, etc.

### BRIEF DESCRIPTION OF THE DRAWINGS

In the drawings, like reference characters generally refer to the same parts throughout the different views. The drawings are not necessarily to scale, emphasis instead generally being placed upon illustrating the exemplary principles of the disclosure. In the following description, various exemplary aspects of the disclosure are described with reference to the following drawings, in which:

FIG. 1 shows an example of wireless-enabled micromotion sensing in the context of nearby static objects and a nearby breathing person;

FIG. 2 illustrates an example flow of wireless-enabled micromotion sensing that evaluates subcarrier channel characteristics to determine the presence of micromotions;

FIG. 3 plots an example set of subcarrier autocorrelations and a combined autocorrelation in an example case were micromotions are not detected;

FIG. 4 plots an example set of subcarrier autocorrelations and a combined autocorrelation in an example case were micromotions are detected;

FIG. 5 illustrates an exemplary schematic drawing of a wireless-enabled micromotion sensing device; and

FIG. 6 depicts an exemplary schematic flow diagram of a method of wireless-enabled micromotion sensing.

### DESCRIPTION

The following detailed description refers to the accompanying drawings that show, by way of illustration, exemplary details and features.

The word “exemplary” is used herein to mean “serving as an example, instance, or illustration”. Any aspect or design described herein as “exemplary” is not necessarily to be construed as preferred or advantageous over other aspects or designs.

Throughout the drawings, it should be noted that like reference numbers are used to depict the same or similar elements, features, and structures, unless otherwise noted.

The phrase “at least one” and “one or more” may be understood to include a numerical quantity greater than or equal to one (e.g., one, two, three, four, [ . . . ], etc., where “[ . . . ]” means that such a series may continue to any higher number). The phrase “at least one of” with regard to a group of elements may be used herein to mean at least one element from the group consisting of the elements. For example, the phrase “at least one of” with regard to a group of elements may be used herein to mean a selection of: one of the listed elements, a plurality of one of the listed elements, a plurality of individual listed elements, or a plurality of a multiple of individual listed elements.

The words “plural” and “multiple” in the description and in the claims expressly refer to a quantity greater than one. Accordingly, any phrases explicitly invoking the aforementioned words (e.g., “plural [elements]”, “multiple [elements]”) referring to a quantity of elements expressly refers to more than one of the said elements. For instance, the phrase “a plurality” may be understood to include a numerical quantity greater than or equal to two (e.g., two, three, four, five, [ . . . ], etc., where “[ . . . ]” means that such a series may continue to any higher number).

The phrases “group (of)”, “set (of)”, “collection (of)”, “series (of)”, “sequence (of)”, “grouping (of)”, etc., in the description and in the claims, if any, refer to a quantity equal to or greater than one, i.e., one or more. The terms “proper subset”, “reduced subset”, and “lesser subset” refer to a subset of a set that is not equal to the set, illustratively, referring to a subset of a set that contains less elements than the set.

The term “data” as used herein may be understood to include information in any suitable analog or digital form, e.g., provided as a file, a portion of a file, a set of files, a signal or stream, a portion of a signal or stream, a set of signals or streams, and the like. Further, the term “data” may also be used to mean a reference to information, e.g., in form of a pointer. The term “data”, however, is not limited to the aforementioned examples and may take various forms and represent any information as understood in the art.

The terms “processor” or “controller” as, for example, used herein may be understood as any kind of technological entity that allows handling of data. The data may be handled according to one or more specific functions executed by the processor or controller. Further, a processor or controller as used herein may be understood as any kind of circuit, e.g., any kind of analog or digital circuit. A processor or a controller may thus be or include an analog circuit, digital circuit, mixed-signal circuit, logic circuit, processor, microprocessor, Central Processing Unit (CPU), Graphics Processing Unit (GPU), Digital Signal Processor (DSP), Field Programmable Gate Array (FPGA), integrated circuit, Application Specific Integrated Circuit (ASIC), etc., or any combination thereof. Any other kind of implementation of the respective functions, which will be described below in further detail, may also be understood as a processor, controller, or logic circuit. It is understood that any two (or more) of the processors, controllers, or logic circuits detailed herein may be realized as a single entity with equivalent functionality or the like, and conversely that any single processor, controller, or logic circuit detailed herein may be realized as two (or more) separate entities with equivalent functionality or the like.

As used herein, “memory” is understood as a computer-readable medium (e.g., a non-transitory computer-readable medium) in which data or information can be stored for retrieval. References to “memory” included herein may thus be understood as referring to volatile or non-volatile memory, including random access memory (RAM), read-only memory (ROM), flash memory, solid-state storage, magnetic tape, hard disk drive, optical drive, 3D XPoint™, among others, or any combination thereof. Registers, shift registers, processor registers, data buffers, among others, are also embraced herein by the term memory. The term “software” refers to any type of executable instruction, including firmware.

Unless explicitly specified, the term “transmit” encompasses both direct (point-to-point) and indirect transmission (via one or more intermediary points). Similarly, the term “receive” encompasses both direct and indirect reception. Furthermore, the terms “transmit,” “receive,” “communicate,” and other similar terms encompass both physical transmission (e.g., the transmission of radio signals) and logical transmission (e.g., the transmission of digital data over a logical software-level connection). For example, a processor or controller may transmit or receive data over a software-level connection with another processor or controller in the form of radio signals, where the physical transmission and reception is handled by radio-layer components such as RF transceivers and antennas, and the logical transmission and reception over the software-level connection is performed by the processors or controllers. The term “communicate” encompasses one or both of transmitting and receiving, i.e., unidirectional or bidirectional communication in one or both of the incoming and outgoing directions. The term “calculate” encompasses both ‘direct’ calculations via a mathematical expression/formula/relationship and ‘indirect’ calculations via lookup or hash tables and other array indexing or searching operations.

As noted above, wireless proximity sensing may involve transmitting wireless signals and then receiving reflections of those signals to make determinations about objects in the environment. For example, a wireless proximity detection system may have a wireless transmitter that may transmit wireless signals and may also have a co-located wireless receiver that may monitor for reflections of the transmitted signals to determine whether an object is nearby. This is known as wireless-based “active sensing.” Wireless proximity detection may have advantages over camera-based systems that require a line of sight to the object and must perform complex image processing to recognize objects. Wireless proximity detection may also have advantages over light detection and ranging (LiDAR) sensors that may require specialized hardware and that also require a line of sight to the objects being detected.

One use of wireless proximity detection may be to detect whether a user is located to a nearby computer. The wireless transceiver (e.g., a Wi-Fi module/controller) of a laptop, for example, may be used in an active sensing mode to transmit signals and then monitor their reflections to determine whether a user is located at the laptop or whether the user is no longer present. If the user is not detected, the laptop may automatically lock a user interface, power-down certain circuitry, etc. If the user is detected, the laptop may wake from low power mode, allow the user interface to be unlocked, etc. However, it may be difficult for wireless active scanning to detect the presence of a person when the person is very still. For example, detection of a person in front of a laptop using conventional wireless active scanning solutions may be faulty, especially when that person is engaged in an activity with minimal or no motion persistent over longer time periods, such as when the user is reading an article or watching a video on the computer screen.

In this type of usage scenario, the wireless active scanning may have difficulty accurately detecting the presence of the user, and as a result, the system confuses absence of the person with detection of the person, which may have serious implications, depending on how the laptop may be using the wireless active sensing. For example, if the wireless active scanning is used for presence detection of a user, where the laptop is designed to lock the laptop upon detecting the absence of the user, the laptop may lock the screen while the user is simply reading an article on the screen. This may lead to user frustration and a poor user experience.

Conventional wireless active sensing may also be subject to false positives, meaning that the system indicates the presence of a user is detected, when in fact, the user is not present and the wireless active sensing was “fooled” by other motion, far away from the laptop. This may be because wireless transceivers are often configured to optimize wireless communication performance and the range of the wireless communication (e.g., optimized for their primary purpose), rather than proximity detection/active sensing (e.g., their secondary purpose). As a result, the wireless sensing system may also be sensitive to objects far away, even though only a small radius (e.g., less than 1.5 meters) around the laptop may be the zone of interest for proximity detection. The implication of this data-communications-focused configuration is that objects in motion away from the laptop (e.g., more than 1.5 meters away) may create a false positive, making the system falsely detect that a user is in front of the laptop, thereby compromising the security of the device.

The disclosed wireless-enabled micromotion sensing system may improve upon wireless active sensing by focusing on the periodicity and/or non-linearity of changes in the channel characteristics over time. Changes in channel characteristics due to static objects (such as a still, relatively motionless person in front of a laptop) may be more subtle such that large variations due to dynamic objects (a moving person) dominate. The disclosed wireless-enabled micromotion sensing system may compensate for this by evaluating the periodicity and/or non-linearity of changes in the channel characteristics over time, rather that just their magnitudes.

As discussed in more detail below, the disclosed wireless-enabled micromotion sensing system may be used to detect very small, periodic motions (referred to herein as a “micromotion”) of nearby objects. For example, the micromotion may be the rise and fall of a person's chest associated with their breathing, the pulsing of a vein associated with the person's heartbeat, the twitching of a muscle, the rate of blinking of the eyes, etc. The wireless-enabled micromotion sensing system may be used to detect the presence of a static/still person, reducing false positives, and better distinguishing between nearby objects and objects far away. Given that many computing devices such as laptops already include wireless modules, the wireless-enabled micromotion sensing system may utilize such hardware without the need for additional hardware components.

Beyond proximity detection (e.g., for sensing whether a user is at their laptop), the wireless-enabled micromotion sensing system may be used in other contexts, such as to detect a person's heartrate or respiration patterns for health monitoring, to detect the subtle vibrations of a piece of equipment (e.g., a refrigerator, industrial equipment, machinery, etc.) over time for determining the predictive maintenance needs of the equipment, expected meantime between failures, lubrication deficiencies, etc. More generally, the disclosed wireless-enabled micromotion sensing system may be used in any context where small, periodic motions may be present.

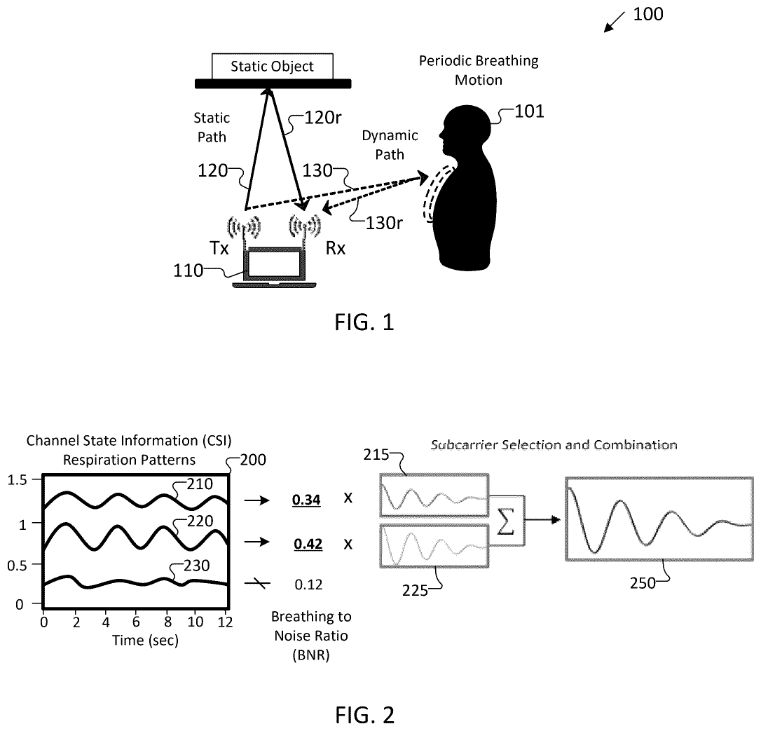

FIG. 1 shows a high-level example of how a wireless-enabled micromotion sensing system 100 may operate to detect, in this example, period breathing motion of a nearby human 101. A wireless device 110 (e.g., a wireless controller/wireless transceiver) may have a transmit (TX) antenna and a receive (RX) antenna that may be used for, respectively, sending and receiving wireless signals. The signals transmitted from the wireless device 110 may be reflected off of nearby objects, including human 101. For example, a signal 120 transmitted from the TX antenna of wireless device 110 may be reflected off of a static object, returning to the RX antenna of wireless device 110 as a reflected signal 120r. As another example signal 130 may be reflected off of the nearby human 101 as a reflected signal 130r. Over time, signals that reflect off of the nearby human 101, may be dynamic due to the micromotions of the human 101, such as the rise and fall of the chest of the human 101.

The transmitted signals for wireless-enabled micromotion sensing may be referred to as probe transmissions on a wireless channel, where the probe transmissions may differentiate themselves from normal wireless communication signals in that they may not carry a data payload and they are not intended for receipt by or communication with a wireless access point. Instead, probe transmissions are meant to be transmitted as non-packet transmissions from a TX antenna so that the reflected signals may be received by the co-located RX antenna.

The wireless-enabled micromotion sensing may monitor—over time—the change in channel characteristics (e.g., channel state information or “CSI”) between the probe transmission and the reflected signal to make determinations about micromotions of nearby objects such as human 101. The change in channel state information may include the change in amplitude and phase between the probe transmission and its reflected signal.

As should be understood, in the context of wireless systems, the wireless channel may be defined by the wireless technology or the wireless standard being used, such as the standards published by the Institute of Electrical and Electronics Engineers (IEEE), known as the IEEE 802.11 wireless standards, including, for example IEEE 802.11-1999, “IEEE Standard for Information Technology-Telecommunications and Information Exchange Between Systems-Local and Metropolitan Area Networks-Specific Requirements-Part 11: Wireless LAN Medium Access Control (MAC) and Physical Layer (PHY) Specifications,” published Jan. 1, 2003. This and following versions are referred to generally throughout as the IEEE 802.11 standards. In the IEEE 802.11 standards, various channel bandwidths may be used, including, as non-limiting examples, 20 MHz, 40 MHz, 80 MHz, 160 MHz, or 320 MHz channels. While these examples are given, the wireless-enabled micromotion sensing is not limited to any particular channel bandwidth. As should be appreciated, wireless transmissions on the channel may be multiplexed into different subcarriers (also called symbols), as is typical in IEEE 802.11 systems. To the extent larger the channel bandwidths are used (for example, 160 MHz and greater), it may be advantageous to divide the channel bandwidth into smaller bandwidth chunks (e.g., a 160 MHz channel may be divided into eight chunks of 20 MHz bands) and choose the best chunk (e.g., the chunk with the highest breath-to-noise ratio (BNR), where BNR is discussed in more detail below). This may further improve the overall system performance by exploiting the larger channel diversity.

In wireless systems such as IEEE 802.11, the channel characteristics may be reported in a CSI report. The CSI report may include information about the state or characteristics of the communication channel associated with that specific subcarrier, including phase and amplitude changes of the channel In addition to phase and amplitude, the CSI report may include estimates or information about several other characteristics of the wireless channel, including the delay spread, the doppler shift, the frequency selectivity, the multipath profile, the channel correlation, the noise power, spatial characteristics, etc. While the examples described herein refer to amplitude and phase channel characteristics, any channel characteristics may be used by the wireless-enabled micromotion sensing system to make determinations about micromotions. In addition, while IEEE 802.11 is used throughout as an example of a wireless device because of its widespread availability, any type of wireless device may be used for transmitting/receiving signals at co-located antennas.

FIG. 2 shows an example plot 200 of changes in channel characteristics for three subcarriers (each subcarrier represented by a corresponding signal 210, 220, and 230) of probe transmissions over time. The wireless-enabled micromotion sensing system (e.g., wireless-enabled micromotion sensing system 100) may determine the extent to which the changes in channel characteristics represent micromotions. For example, the wireless-enabled micromotion sensing system may determine a breathing-to-noise ratio (BNR) of each subcarrier to determine whether it exhibits periodic micromotions indicative of a breathing person (e.g., a breathing frequency range of about 10-37 breaths per minute or 0.1 to 1.0 Hertz).

BNR may be understood as the ratio of energy in the breathing frequency range vs. the total energy, which estimates how well a given signal (e.g., of the subcarrier) is indicative a breathing micromotion. To calculate BNR, the wireless-enabled micromotion sensing system may apply a filter (e.g., a Savitzky-Golay filter (e.g., with an order of 3 and a length of 20)) to each subcarrier signal, perform a frequency decomposition on each subcarrier signal (e.g., a fast Fourier transform (FFT)), and then determine the energy level within the frequency range of interest for the micromotion (e.g., the breathing frequency range for breathing micromotions, the vibration frequency range for industrial machine monitoring, the pulse range for heartrate monitoring, etc.) divided by the total energy. A higher BNR indicates a higher likelihood that the subcarrier is relevant to micromotion, whereas lower BNRs may indicate static motion or motions outside of the frequency range of interest. As should be appreciated, any type of filtering may be performed using any filter of any length and order, and the Savitzky-Golay filter is just one example. Similarly, and type of frequency decomposition may be used to transform the signal data in the time domain to a representation in the frequency domain, and an FFT is just one example.

The wireless-enabled micromotion sensing system may then rank/omit the subcarriers based on their relative BNR and/or based on a predefined criterion (e.g., only include the five highest subcarriers, only include subcarriers that are above a threshold BNR, etc.). With reference to FIG. 2, the BNR for signal 210 is 0.34, the BNR for signal 220 is 0.42, and the BNR for signal 230 is 0.12. Thus, the wireless-enabled micromotion sensing system may decide to include the top two subcarriers (represented by signal 210 and signal 220) while omitting the subcarrier with the lowest BNR (represented by signal 230).

The wireless-enabled micromotion sensing system may then compute the autocorrelation of each subcarrier signal (e.g., for the subcarriers that satisfy the predefined criterion) and then combine the individual autocorrelations (for each subcarrier) into a combined autocorrelation. To generate the combined autocorrelation, the individual autocorrelations for each subcarrier may be weighted by its corresponding BNR. In other words, the individual subcarrier autocorrelation is first multiplied by its BNR (e.g., a number from 0 to 1) before being added into the combined autocorrelation in order to give higher weight to more relevant subcarriers and lower weight to less relevant subcarriers. In the example of FIG. 2, signal 210 would be multiplied by its BNR of 0.34 while signal 220 would be multiplied by its BNR of 0.42. Signal 215 shows the autocorrelation of signal 210 multiplied by its BNR and signal 225 shows the autocorrelation of signal 225 multiplied by its BNR. These are added together to form the combined autocorrelation 250.

To the extent the first peaks of the individual subcarrier autocorrelations (e.g., autocorrelation 215, 225) align with the combined autocorrelation (e.g., combined autocorrelation 250), this may indicate that a nearby human is breathing (or, more generally, that the micromotion of interest is occurring). Thus, the wireless-enabled micromotion sensing system may determine whether the micromotion of interest is occurring based on the extent of alignment of the first peaks of the subcarrier autocorrelations to the first peak of the combined autocorrelation (“first-peak alignment”).

The first-peak alignment may be calculated using the following function:

$${t}^{FpAgr} = 1 - ( \frac{1}{{len}_{C A}} {\sum }_{n = 1} \vert {Fp}_{n} - {Fp}_{C A} \vert )$$

In the above equation, Fpₙ is the autocorrelation first peak location of subcarrier n, and variables Fp_{CA} and len_{CA} are the first peak location and length of the combined autocorrelation respectively. The result is the mean of the deltas between each subcarrier autocorrelation first peak and the combined autocorrelation first peak normalized over the length of the window then subtracted from one.

In the case of no micromotion, the wireless-enabled micromotion sensing system may compute the subcarrier autocorrelations based on changing channel characteristics (e.g., in amplitude and phase) which are random and represent noise (e.g., due to the absence of any periodic micromotions, such as breathing). As a result, the wireless-enabled micromotion sensing system may determine there is poor alignment between the curves and this value will trend towards zero, suggesting no micromotion is present. In the case where micromotion exists, the subcarrier autocorrelations are computed on the period patterns in the changing channel characteristics associated with the micromotion (e.g., breathing patterns). As a result, the wireless-enabled micromotion sensing system may determine there is good subcarrier alignment as the autocorrelations for each subcarrier should represent the same periodic micromotion, therefore having a larger value for first-peak alignment and suggesting presence of the micromotion.

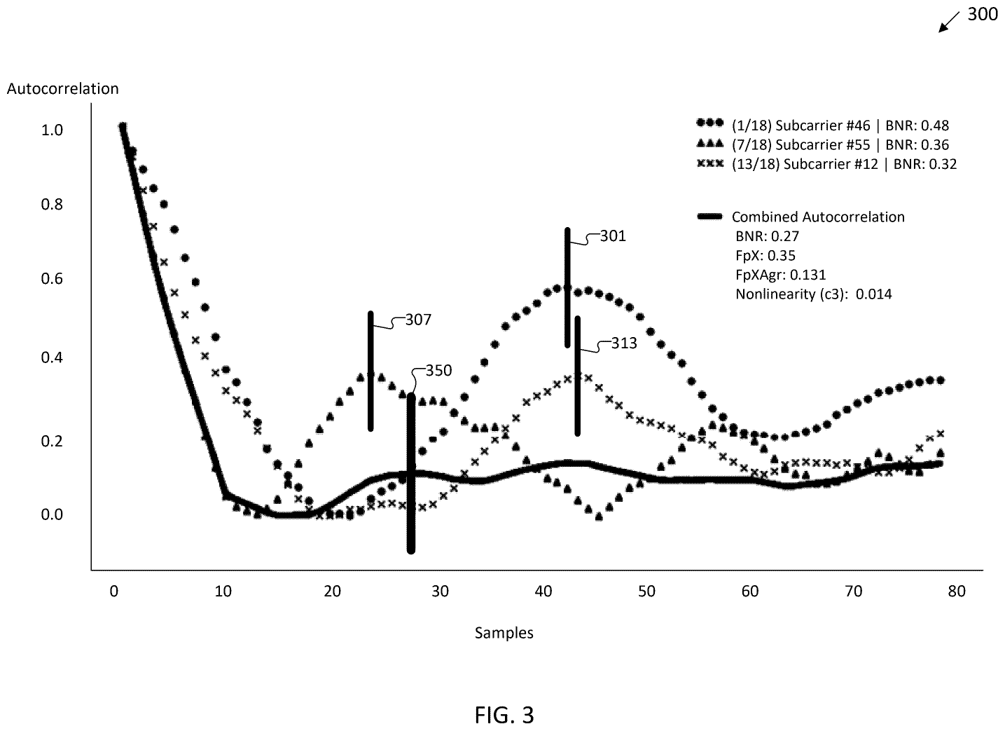

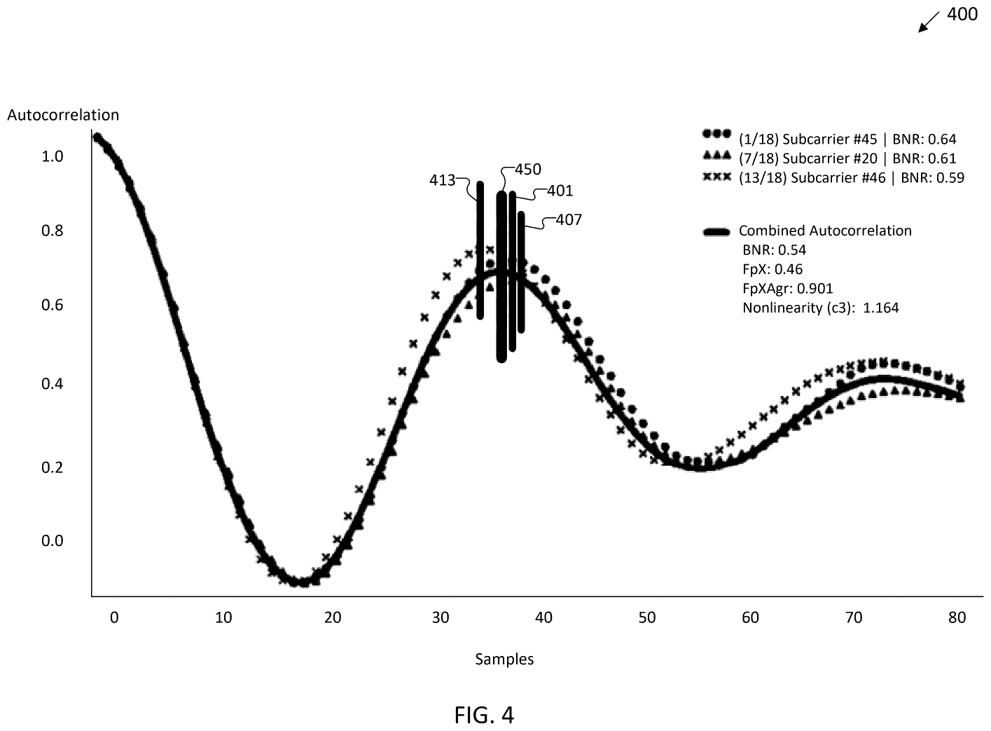

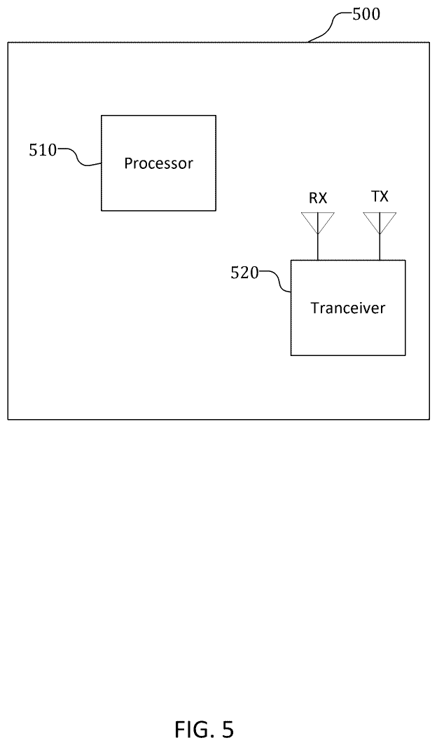

Referring to FIGS. 3 and 4, these plot example subcarrier correlations along with the combined autocorrelation of multiple subcarriers of samples over time. The extent of alignment of the first peak of each subcarrier correlations to the first peak of the combined autocorrelation may indicate whether the micromotion of interest is occurring. In FIG. 3, for example, plot 300 shows the autocorrelations of three subcarriers (subcarrier #46 with a BNR of 0.48, subcarrier #55 with a BNR of 0.36, and subcarrier #12 with a BNR of 0.32) are plotted along with the combined autocorrelation. As noted earlier, these three subcarriers may be subcarriers selected by the wireless-enabled micromotion sensing system for inclusion in the combined autocorrelation based on the predetermined criteria (e.g., they met a threshold, had the highest relative values, etc.). The first peaks of each subcarrier autocorrelation and of the combined autocorrelation have been marked in plot 300, where peak 301 is for the autocorrelation of subcarrier #46, peak 307 is for the autocorrelation of subcarrier #55, peak 313 is for the autocorrelation of subcarrier #12, and peak 350 is for the combined autocorrelation. As can be seen in plot 300, there is relatively poor alignment of the individual subcarrier peaks 307, 301, and 313 to the combined peak 350. Thus, the wireless-enabled micromotion sensing system may determine that there is no micromotion occurring.

This is contrast to FIG. 4, where plot 400 shows the autocorrelations of another set of subcarriers (subcarrier #46 with a BNR of 0.64, subcarrier #20 with a BNR of 0.61, and subcarrier #46 with a BNR of 0.59) plotted along with the combined autocorrelation in another situation. In this situation, there is substantial alignment among the first peaks of each subcarrier autocorrelation (401, 407, and 413) to the first peak of the combined autocorrelation (450). Thus, the wireless-enabled micromotion sensing system may determine that the micromotion is indeed occurring.

In addition to first-peak alignment, the wireless-enabled micromotion sensing system may compute the extent of nonlinearity of the combined autocorrelation. The higher the measure of non-linearity, the more likely a micromotion is detected. For example, the wireless-enabled micromotion sensing system may compute the c3 statistic as a measure of nonlinearity, where c3 is a higher order cumulant/autocovariance measure that may be sensitive to any deviation from noise and helps detect small changes in subcarrier agreement. The c3 statistic may be calculated based on the following function, where x is the combined autocorrelation and lag=2:

$${t}^{c 3} = \frac{1}{n - 2 lag} {\sum }_{i = 1}^{n - 2 lag} {x}_{i + 2 lag} \cdot {x}_{i + lag} \cdot {x}_{i}$$

In the case of no presence, the combined autocorrelation approaches the Dirac delta function, which is linear in nature, having constant output for all inputs and therefore decreasing the nonlinearity value. In the case of presence, agreement between subcarriers results in a combined autocorrelation with periodic, sinusoidal shape with amplitude decay, which are characteristics of nonlinear systems and therefore increasing the nonlinearity value. As shown in FIG. 3, the nonlinearity value is a relatively low value of 0.014, which may be indicative of no occurrence of micromotion, whereas in FIG. 4, the nonlinearity value is a relatively high value of 1.164, which may be indicative of occurrence of micromotion. As should be understood, the wireless-enabled micromotion sensing system may determine whether micromotion is occurrent based on first-peak alignment, the linearity, and/or both (e.g., whether each metric satisfies a predefined criterion, by weighting each metric, etc.).

FIG. 5 is a schematic drawing illustrating a device 500 for wireless-enabled micromotion sensing. The device 500 may include any of the features with respect to the wireless-enabled micromotion sensing systems discussed above and any of FIGS. 1-4. FIG. 5 may be implemented as a device, a system, a method, and/or a computer readable medium that, when executed, performs the features of the wireless-enabled micromotion sensing system described above. It should be understood that device 500 is only an example, and other configurations may be possible that include, for example, different components or additional components.

Device 500 includes a processor 510. Processor 510 of device 500 is configured to cause a transmit antenna (e.g., as part of transceiver 520) to wirelessly transmit a series of probe transmissions. Processor 510 is also configured to cause a receive antenna (e.g., that is co-located with the transmit antenna) (e.g., also part of transceiver 520) to wirelessly receive a series of reflected signals from the probe transmissions, each reflected signal of the series of reflected signals having a corresponding transmission of the series of probe transmissions. Processor 510 is also configured to determine a characteristic dataset comprising a change in a channel characteristic over time as between the series of reflected signals and the series of probe transmissions. Processor 510 is also configured to determine based on the characteristic dataset whether the series of reflected signals is indicative of a periodic micromotion of an object.

Furthermore, in addition to or in combination with any of the features described in this or the preceding paragraph with respect to device 500, the change in the channel characteristic may include a channel state information (CSI) report. Furthermore, in addition to or in combination with any of the features described in this or the preceding paragraph, the channel state information (CSI) report may include a change in amplitude and/or phase of a subcarrier of a wireless channel of the series of probe transmissions. Furthermore, in addition to or in combination with any of the features described in this or the preceding paragraph, the object may include a person, the periodic micromotion may include a breathing motion of the person. Furthermore, in addition to or in combination with any of the features described in this or the preceding paragraph, the object may include a person, the periodic micromotion may include a heartrate of the person. Furthermore, in addition to or in combination with any of the features described in this or the preceding paragraph, the object may include a machine, the periodic micromotion may include a vibration of the machine.

Furthermore, in addition to or in combination with any of the features described in this or the preceding two paragraphs with respect to device 500, processor 510 may be further configured to determine whether the series of reflected signals is indicative of the periodic micromotion based on a learning model that relates changes in channel characteristics over time to a probability that objects exhibit the periodic micromotion. Furthermore, in addition to or in combination with any of the features described in this or the preceding two paragraphs, the series of probe transmissions may include a plurality of non-data packet transmissions over time, interleaved among regular data packet transmissions of a wireless system. Furthermore, in addition to or in combination with any of the features described in this or the preceding two paragraphs, the wireless system may include a wireless local area network system (e.g., a Wi-Fi system). Furthermore, in addition to or in combination with any of the features described in this or the preceding two paragraphs, processor 510 may be configured to determine whether the object is proximate to the transmit antenna based on the change in the channel characteristic over time.

Furthermore, in addition to or in combination with any of the features described in this or the preceding three paragraphs, processor 510 may be configured to control, based on whether the series of reflected signals is indicative of the periodic micromotion, a locking or an unlocking of a user interface of a computing platform. Furthermore, in addition to or in combination with any of the features described in this or the preceding three paragraphs, the change in the channel characteristic may include a change in a phase and/or an amplitude as between the reflected signal and its corresponding probe transmission. Furthermore, in addition to or in combination with any of the features described in this or the preceding three paragraphs, processor 510 may be further configured to cause the transmit antenna to wirelessly transmit the series of probe transmissions and to cause the receive antenna to wirelessly receive the series of reflected signals on a wireless channel including a plurality of subcarriers, wherein the channel characteristic may include a subchannel characteristic for each subcarrier of the plurality of subcarriers of the wireless channel, wherein the characteristic dataset may include a subcarrier dataset for each subcarrier of the plurality of subcarriers.

Furthermore, in addition to or in combination with any of the features described in this or the preceding four paragraphs, processor 510 may be configured to determine a breathing-to-noise ratio (BNR) of each subcarrier dataset based on its corresponding subchannel characteristic. Furthermore, in addition to or in combination with any of the features described in this or the preceding four paragraphs, the BNR may include a ratio of energy in a portion of a bandwidth of the subcarrier over a total energy in the bandwidth of the subcarrier. Furthermore, in addition to or in combination with any of the features described in this or the preceding four paragraphs, the portion of a bandwidth may include a breathing frequency range including about 0.1 to 1.0 Hz. Furthermore, in addition to or in combination with any of the features described in this or the preceding four paragraphs, a bandwidth of the wireless channel may include 20 MHz, 40 MHz, 80 MHz, 160 MHz, or 320 MHz. Furthermore, in addition to or in combination with any of the features described in this or the preceding four paragraphs, processor is configured to smooth the characteristic dataset with a filter. Furthermore, in addition to or in combination with any of the features described in this or the preceding four paragraphs, the filter may include a Savitzky-Golay filter (e.g., with an order of 3 and a length of 20).

Furthermore, in addition to or in combination with any of the features described in this or the preceding five paragraphs, processor 510 is configured to determine the BNR for each subcarrier dataset based on a frequency decomposition (e.g., a fast Fourier transform (FFT)) of the subcarrier dataset. Furthermore, in addition to or in combination with any of the features described in this or the preceding five paragraphs, the frequency decomposition may include a set of bins, wherein the BNR may include an energy of one or more bins of the set that has a highest energy among the set divided by a total energy of the set. Furthermore, in addition to or in combination with any of the features described in this or the preceding five paragraphs, the set of bins may be defined by an order and a length of the frequency decomposition, wherein each bin may contain an amplitude and a phase from the subcarrier dataset in a frequency range of the bin. Furthermore, in addition to or in combination with any of the features described in this or the preceding five paragraphs, processor 510 may further be configured to filter out an insignificant subcarrier dataset from the subcarrier datasets based on whether the BNR of the insignificant subcarrier dataset satisfies a predefined criterion (e.g. higher than a threshold BNR level).

Furthermore, in addition to or in combination with any of the features described in this or the preceding six paragraphs, processor 510 may be configured to combine an autocorrelation of each of the subcarrier datasets into a combined autocorrelation. Furthermore, in addition to or in combination with any of the features described in this or the preceding six paragraphs, processor 510 may be configured to combine the autocorrelation of each of the subcarrier datasets, weighted by its BNR. Furthermore, in addition to or in combination with any of the features described in this or the preceding six paragraphs, the combined autocorrelation may include a weighted sum of each autocorrelation, where each autocorrelation may be weighted by a weight that may be related to its corresponding BNR. Furthermore, in addition to or in combination with any of the features described in this or the preceding six paragraphs, the processor 510 configured to determine whether the series of reflected signals is indicative of the periodic micromotion may include processor 510 configured to determine whether the combined autocorrelation satisfies a predefined criterion.

Furthermore, in addition to or in combination with any of the features described in this or the preceding seven paragraphs, the predefined criterion may include an extent of alignment among a first peak of each autocorrelation in the combined autocorrelation. Furthermore, in addition to or in combination with any of the features described in this or the preceding seven paragraphs, the predefined criterion may include an extent to which a first peak in the combined autocorrelation exceeds an average of combined autocorrelation. Furthermore, in addition to or in combination with any of the features described in this or the preceding seven paragraphs, the predefined criterion may include an extent of linearity of the combined autocorrelation. Furthermore, in addition to or in combination with any of the features described in this or the preceding seven paragraphs,

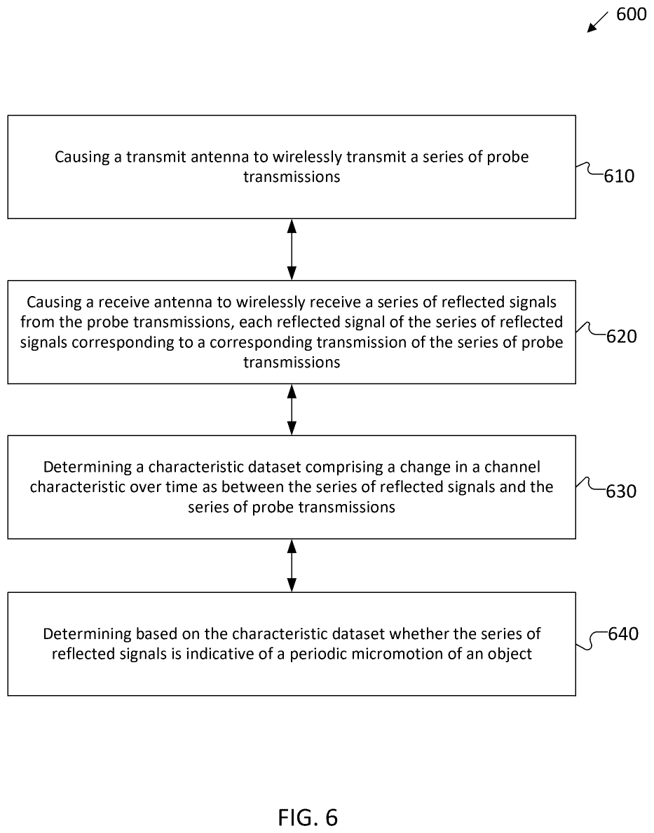

FIG. 6 depicts a schematic flow diagram of a method 600 for wireless-enabled micromotion sensing. Method 600 may implement any of the features with respect to the wireless-enabled micromotion sensing systems discussed above and/or FIGS. 1-5. Method 600 includes, in 610, causing a transmit antenna to wirelessly transmit a series of probe transmissions. Method 600 also includes, in 620, causing a (co-located) receive antenna to wirelessly receive a series of reflected signals from the probe transmissions, each reflected signal of the series of reflected signals having a corresponding transmission of the series of probe transmissions. Method 600 also includes, in 630, determining a characteristic dataset comprising a change in a channel characteristic over time as between the series of reflected signals and the series of probe transmissions. Method 600 also includes, in 640, determining based on the characteristic dataset whether the series of reflected signals is indicative of a periodic micromotion of an object.

In the following, various examples are provided that may include one or more aspects described with reference to the wireless-enabled micromotion sensing systems discussed above and/or any of FIGS. 1-6. The examples provided in relation to the devices may apply also to the described method(s), and vice versa.

Example 1 is a device including a transceiver and processor. The processor configured to cause a transmit antenna of the transceiver to wirelessly transmit a series of probe transmissions. The processor is also configured to cause a (e.g., co-located) receive antenna of the transceiver to wirelessly receive a series of reflected signals from the probe transmissions, each reflected signal of the series of reflected signals having a corresponding transmission of the series of probe transmissions. The processor is also configured to determine a characteristic dataset comprising a change in a channel characteristic over time as between the series of reflected signals and the series of probe transmissions. The processor is also configured to determine based on the characteristic dataset whether the series of reflected signals is indicative of a periodic micromotion of an object.

Example 2 is the device of example 1, wherein the change in the channel characteristic includes a channel state information (CSI) report.

Example 3 is the device of example 2, wherein the channel state information (CSI) report includes a change in amplitude and/or phase of a subcarrier of a wireless channel of the series of probe transmissions.

Example 4 is the device of any one of examples 1 to 3, wherein the object includes a person, wherein the periodic micromotion includes a breathing motion of the person.

Example 5 is the device of any one of examples 1 to 4, wherein the object includes a person, wherein the periodic micromotion includes a heartrate of the person.

Example 6 is the device of any one of examples 1 to 5, wherein the object includes a machine, wherein the periodic micromotion includes a vibration of the machine.

Example 7 is the device of any one of examples 1 to 6, wherein the processor is further configured to determine whether the series of reflected signals is indicative of the periodic micromotion based on a learning model that relates changes in channel characteristics over time to a probability that objects exhibit the periodic micromotion.

Example 8 is the device of any one of examples 1 to 7, wherein the series of probe transmissions include a plurality of non-data packet transmissions over time, interleaved among regular data packet transmissions of a wireless system.

Example 9 is the device of example 8, wherein the wireless system includes a wireless local area network system (e.g., a Wi-Fi system).

Example 10 is the device of any one of examples 1 to 9, determine whether the object is proximate to the transmit antenna based on the change in the channel characteristic over time.

Example 11 is the device of any one of examples 1 to 10, wherein the processor is configured to control, based on whether the series of reflected signals is indicative of the periodic micromotion, a locking or an unlocking of a user interface of a computing platform.

Example 12 is the device of any one of examples 1 to 11, wherein the change in the channel characteristic includes a change in a phase and/or an amplitude as between the reflected signal and its corresponding probe transmission.

Example 13 is the device of any one of examples 1 to 12, wherein the processor is further configured to cause the transmit antenna to wirelessly transmit the series of probe transmissions and to cause the receive antenna to wirelessly receive the series of reflected signals on a wireless channel including a plurality of subcarriers, wherein the channel characteristic includes a subchannel characteristic for each subcarrier of the plurality of subcarriers of the wireless channel, wherein the characteristic dataset includes a subcarrier dataset for each subcarrier of the plurality of subcarriers.

Example 14 is the device of example 13, wherein the processor is configured to determine a breathing-to-noise ratio (BNR) of each subcarrier dataset based on its corresponding subchannel characteristic.

Example 15 is the device of example 14, wherein the BNR includes a ratio of energy in a portion of a bandwidth of the subcarrier over a total energy in the bandwidth of the subcarrier.

Example 16 is the device of example 15, wherein the portion of a bandwidth includes a breathing frequency range including about 0.1 to 1.0 Hz.

Example 17 is the device of any one of examples 13 to 16, wherein a bandwidth of the wireless channel includes 20 MHz, 40 MHz, 80 MHz, 160 MHz, or 320 MHz.

Example 18 is the device of any one of examples 1 to 17, wherein the processor is configured to smooth the characteristic dataset with a filter.

Example 19 is the device of example 18, wherein the filter includes a Savitzky-Golay filter (e.g., with an order of 3 and a length of 20).

Example 20 is the device of any one of examples 15 to 19, wherein the processor is configured to determine the BNR for each subcarrier dataset based on a frequency decomposition (e.g., a fast Fourier transform (FFT)) of the subcarrier dataset.

Example 21 is the device of example 20, wherein the frequency decomposition includes a set of bins, wherein the BNR includes an energy of one or more bins of the set that has a highest energy among the set divided by a total energy of the set.

Example 22 is the device of example 21, wherein the set of bins are defined by an order and a length of the frequency decomposition, wherein each bin contains an amplitude and a phase from the subcarrier dataset in a frequency range of the bin.

Example 23 is the device of any one of examples 14 to 22, wherein the processor is further configured to filter out an insignificant subcarrier dataset from the subcarrier datasets based on whether the BNR of the insignificant subcarrier dataset satisfies a predefined criterion (e.g. higher than a threshold BNR level).

Example 24 is the device of any one of examples 14 to 23, wherein the processor is configured to combine an autocorrelation of each of the subcarrier datasets into a combined autocorrelation.

Example 25 is the device of example 24, wherein the processor is configured to combine the autocorrelation of each of the subcarrier datasets, weighted by its BNR.

Example 26 is the device of example 25, wherein the combined autocorrelation includes a weighted sum of each autocorrelation, where each autocorrelation is weighted by a weight that is related to its corresponding BNR.

Example 27 is the device of any one of examples 25 to 26, wherein the processor configured to determine based on the characteristic dataset whether the series of reflected signals is indicative of a periodic micromotion of an object includes the processor configured to determine whether the combined autocorrelation satisfies a predefined criterion.

Example 28 is the device of example 27, wherein the predefined criterion includes an extent of alignment among a first peak of each autocorrelation in the combined autocorrelation.

Example 29 is the device of example 27, wherein the predefined criterion includes an extent to which a first peak in the combined autocorrelation exceeds an average of combined autocorrelation.

Example 30 is the device of example 27, wherein the predefined criterion includes an extent of linearity of the combined autocorrelation.

While the disclosure has been particularly shown and described with reference to specific aspects, it should be understood by those skilled in the art that various changes in form and detail may be made therein without departing from the spirit and scope of the disclosure as defined by the appended claims. The scope of the disclosure is thus indicated by the appended claims and all changes, which come within the meaning and range of equivalency of the claims, are therefore intended to be embraced.

1. A device comprising:
a transceiver; and
a processor configured to:
cause a transmit antenna of the transceiver to wirelessly transmit a series of probe transmissions;
cause a receive antenna of the transceiver to wirelessly receive a series of reflected signals from the series of probe transmissions, each reflected signal of the series of reflected signals corresponding to a corresponding transmission of the series of probe transmissions;
determine a characteristic dataset comprising a change in a channel characteristic over time as between the series of reflected signals and the series of probe transmissions; and
determine based on the characteristic dataset whether the series of reflected signals is indicative of a periodic micromotion of an object.

2. The device of claim 1, wherein the change in the channel characteristic comprises a channel state information (CSI) report.

3. The device of claim 2, wherein the CSI report comprises a change in amplitude and/or phase of a subcarrier of a wireless channel of the series of probe transmissions.

4. The device of claim 1, wherein the processor is configured to control, based on whether the series of reflected signals is indicative of the periodic micromotion, a locking or an unlocking of a user interface of a computing platform.

5. The device of claim 1, wherein the change in the channel characteristic comprises a change in a phase or an amplitude as between the reflected signal and its corresponding probe transmission.

6. The device of claim 1, wherein the processor is further configured to cause the transmit antenna to wirelessly transmit the series of probe transmissions and to cause the receive antenna to wirelessly receive the series of reflected signals on a wireless channel comprising a plurality of subcarriers, wherein the channel characteristic comprises a subchannel characteristic for each subcarrier of the plurality of subcarriers of the wireless channel, wherein the characteristic dataset comprises a subcarrier dataset for each subcarrier of the plurality of subcarriers.

7. The device of claim 6, wherein the processor is configured to determine a breathing-to-noise ratio (BNR) of each subcarrier dataset based on its corresponding subchannel characteristic, wherein the BNR comprises a ratio of energy in a portion of a bandwidth of the subcarrier over a total energy in the bandwidth of the subcarrier.

8. The device of claim 7, wherein the processor is configured to determine the BNR for each subcarrier dataset based on a frequency decomposition of the subcarrier dataset, wherein the frequency decomposition comprises a set of bins, wherein the BNR comprises an energy of one or more bins of the set that has a highest energy among the set divided by a total energy of the set.

9. The device of claim 8, wherein the set of bins are defined by an order and a length of the frequency decomposition, wherein each bin contains an amplitude and a phase from the subcarrier dataset in a frequency range of the bin.

10. The device of claim 7, wherein the processor is further configured to filter out an insignificant subcarrier dataset from the subcarrier datasets based on whether the BNR of the insignificant subcarrier dataset satisfies a predefined criterion.

11. The device of claim 6, wherein the processor is configured to combine an autocorrelation of each of the subcarrier datasets into a combined autocorrelation.

12. The device of claim 11, wherein the combined autocorrelation comprises a weighted sum of each autocorrelation, where each autocorrelation is weighted by a weight that is related to its corresponding BNR.

13. The device of claim 11, wherein the processor configured to determine whether the series of reflected signals is indicative of the periodic micromotion comprises the processor configured to determine whether the combined autocorrelation satisfies a predefined criterion.

14. The device of claim 13, wherein the predefined criterion comprises:
an extent of alignment among a first peak of each autocorrelation in the combined autocorrelation; or
an extent of linearity of the combined autocorrelation.

15. The device of claim 1, wherein the object comprises a person, wherein the periodic micromotion comprises a breathing motion of the person, a heartrate of the person, or an eye-blink rate of the person.

16. The device of claim 1, wherein the processor is further configured to determine whether the series of reflected signals is indicative of the periodic micromotion based on an output of a learning model, wherein the learning model relates changes in channel characteristics over time to a probability that objects exhibit the periodic micromotion.

17. A wireless-enabled micromotion sensing system comprising:
a means for transmitting a series of probe transmissions;
a means for receiving a series of reflected signals from the series of probe transmissions, each reflected signal of the series of reflected signals corresponding to a corresponding transmission of the series of probe transmissions;
a means for determining a characteristic dataset comprising a change in a channel characteristic over time as between the series of reflected signals and the series of probe transmissions; and
a means for determining based on the characteristic dataset whether the series of reflected signals is indicative of a periodic micromotion of an object.

18. The wireless-enabled micromotion sensing system of claim 17, wherein the object comprises a person, wherein the periodic micromotion comprises a breathing motion of the person, a heartrate of the person, or an eye-blink rate of the person.

19. A non-transitory computer-readable medium comprising instructions that, when executed by one or more processors, cause the one or more processors to:
cause a transmit antenna to wirelessly transmit a series of probe transmissions;
cause a receive antenna to wirelessly receive a series of reflected signals from the probe transmissions, each reflected signal of the series of reflected signals corresponding to a corresponding transmission of the series of probe transmissions;
determine a characteristic dataset comprising a change in a channel characteristic over time as between the series of reflected signals and the series of probe transmissions; and
determine based on the characteristic dataset whether the series of reflected signals is indicative of a periodic micromotion of an object.

20. The non-transitory computer-readable medium of claim 19, wherein the instructions also cause the one or more processors to determine whether the series of reflected signals is indicative of the periodic micromotion based on an output of a learning model, wherein the learning model relates changes in channel characteristics over time to a probability that objects exhibit the periodic micromotion.
